(19) United States
(12) Patent Application Publication
(10) Pub. No.: US 2018/0333305 A1
(43) Pub. Date: Nov. 22, 2018

(54) Abdominal Area Dry Protector

(71) Applicant: Misty J. Bennett, Eagle Mountain, UT (US)

(72) Inventor: Misty J. Bennett, Eagle Mountain, UT (US)

(21) Appl. No.: 15/980,580

(22) Filed: May 15, 2018

Related U.S. Application Data
(60) Provisional application No. 62/507,143, filed on May 16, 2017.

(51) Int. Cl. A61F 13/14; A61F 13/15; A61F 13/64

(52) U.S. Cl. CPC A61F 13/148; A61F 13/15203; A61F 2013/00319; A61F 2013/1556; A61F 13/64

(57) ABSTRACT

An apparatus for keeping dry a portion of a person's skin, including a belt portion having an outer side and an inner side, configured for placement of the inner side against an abdominal area of a person's body to prevent water from passing between the inner side and the abdominal area, and also including a body portion configured to cover at least a portion of the abdominal area. The body portion may include a body top end forming a first opening sized to receive a portion of the abdominal area, and include a body bottom end having at least one second opening sized to receive a portion of the person's body, and a body middle connecting the body top end and the body bottom end. Also, a method for keeping dry a target portion of a person's skin using an apparatus having a belt portion and a body portion.

### CROSS REFERENCES

The present application for patent is a continuation of U.S. Patent Application No. 62/507,143 by Bennett entitled “Abdominal Area Dry Protector,” filed May 16, 2017, and which is hereby incorporated by reference in its entirety.

### TECHNICAL FIELD

This disclosure relates to the field of protecting areas of the body from liquids. More specifically, the disclosure relates to preventing areas of the skin having incisions—particularly in the abdominal area and below—from contact with water, for example, while showering.

### BACKGROUND

After a surgery or other procedure, patients are often asked to keep an incision (or multiple incisions) and/or drain dry for a period of time. In some cases, this period may be 24-48 hours and in other cases, not until a wound is fully healed. Not following this advice and allowing an incision to get wet may result in infection and/or damaging the incision. However, washing the rest of the body while keeping the incision dry—for example, while showering—can be challenging. Many existing apparatuses and methods do not adequately prevent shower water from intruding into a wounded area. For example, some persons resort to placing a plastic wrap, cling film, or even a trash bag around a wounded area, and then taping around the edges. Due to the time-consuming nature and ineffectiveness of these and other methods and apparatuses in preventing incision areas from getting wet while showering, patients instead carefully wash themselves using sponges or wet wipes, which may also be potentially time-consuming and generally less efficient or refreshing than showering. Moreover, so-called “waterproof” dressings, for a variety of reasons, may be prohibitively expensive, especially for incisions that take longer to heal and where repeated showering is desired, and in some instance may similarly not prevent water from entering a wounded area.

### SUMMARY OF THE INVENTION

The foregoing and other features, utilities and advantages of the invention will be apparent from the following more particular description of a preferred embodiment of the invention as illustrated in the accompanying drawings.

In one embodiment, described herein is an apparatus for keeping dry a portion of a person's skin. The apparatus may include a belt portion having an outer side and an inner side, and be configured for placement of the inner side against an abdominal area of a person's body to prevent water from passing between the inner side and the abdominal area. The belt portion may include waterproof material. The apparatus may also include a body portion configured to cover at least a portion of the abdominal area. The body portion may include a body top end forming a first opening sized to receive a portion of the abdominal area. The body top end may also be fastenable to the belt portion adjacent to the first opening to form a liquid impermeable connection. The body portion may also include a body bottom end having at least one second opening sized to receive a portion of the person's body, and a body middle connecting the body top end and the body bottom end, and comprising liquid-impermeable material.

In some examples of the apparatus, the body portion may be tubular shaped and the portion of the person's body the second opening may be sized to receive may be another portion of the abdominal area. In some examples, the belt portion and the body portion may be further configured for placement against the abdominal area at at least one of: a torso, a midriff, a waist, and an abdomen. In some examples, the belt portion may include a distal end and a proximal end opposite from the distal end and a fastener for fastening the distal end to the proximate end. In some examples, the belt portion may include elastic properties along an axis defined by a length of the belt between the distal end and the proximal end.

In some examples of the apparatus, the body top end may be fastenable to the belt portion along the body top end opening to form a liquid impermeable connection therebetween. In some examples, the belt portion may be fastenable so that a belt top end of fastened belt portion lies a first distance above the body top end and a belt bottom end lies a second distance below the body top end. In some examples, the belt portion may removably fasten to the body portion.

In some examples of the apparatus, the belt portion may include a first material and a second material distinct from the first material. In some examples, the first material may be associated with the outer side of the belt portion, and the second material may be associated with the inner side of the belt portion. In some examples, the first material may be associated with the outer surface of the belt portion, and the second material may be associated with an interior of the belt portion.

In some examples of the apparatus, the waterproof material may include synthetic rubber. In some examples, the synthetic rubber may include polychloroprene. In some examples, the polychloroprene may include neoprene, and in some examples 100% neoprene. In some examples, the belt portion may have an exterior that includes the waterproof neoprene and an interior that includes rubber. In some examples, the neoprene may be bonded with polyester. In some examples, the belt portion may include spandex. In some examples, the belt portion comprises neoprene bonded sponge waterproof wetsuit fabric.

In some examples, the belt portion may be configured to apply a force against a skin surface of the abdominal area to prevent water from passing between the inner side of the belt portion and the skin surface of the abdominal area.

In some examples, the belt portion may have a distal end and a proximal end opposite from the distal end, and the belt portion may be configured to expand and contract in the both the direction of the distal end and in the direction of the proximal end. In some examples, the apparatus may also include a fastener to fasten the distal end to the proximate end while the belt portion is wrapped around the abdominal area. In some examples, the belt portion may have a length between the distal end and the proximate end fastened to the distal end, and the length may be adjustable for wrapping the belt portion around different sized abdominal areas. In some examples, the length may be in the range of about 40-75 inches. In some examples, the fastener may include hook and loop fasteners.

In some examples, the belt portion may include a belt top end and a belt bottom end and a height separating the belt top end from the belt bottom end. In some examples, the body top end may be fastenable to the belt portion along the body top end opening to form a liquid impermeable connection therebetween. In some examples, the body top end may be fastenable to the belt bottom end to form the liquid impermeable connection therebetween. In some examples, the body top end may be configured to fasten to the inner side of the belt portion so that the belt top end lies a first distance above the body top end, and the belt bottom end lies a second distance below the body top end. In some examples, the first distance may be in the range of about 0.5 inches to about 3 inches and the second distance may be in the range of about 0.5 inches to about 3 inches. In some examples, the body top end may include an elastic material. In some examples, the belt portion may be configured for placement of at least part of the inner side of the belt top end against the abdominal area to prevent water from passing between the inner side of the belt top end and the abdominal area.

In some examples, the belt portion may be configured to cinch the body portion. In some examples, the belt portion may be configured for placement of the inner side against the abdominal area at at least one of: a torso, a midriff, a waist, and an abdomen of the person.

In some examples, the body portion may be tubular shaped. In some examples, the liquid-impermeable material may include ripstop nylon fabric. In some examples, the body bottom end may include elastic material. In some examples, the body bottom end may have two openings to fit each of the legs of the person therethrough. In some examples, the body portion may include polyurethane laminate.

In some examples, the belt portion may be removably fastenable to the body portion. In some examples, the belt portion and the body portion may include hook and loop fasteners for removably fastening.

In some examples, the belt portion may have a thickness in the range of about 0.1 millimeters to about 7 millimeters. In some examples, the belt portion may have the thickness in the range of about 0.25 millimeters to about 3 millimeters.

A further embodiment is directed to a method for keeping dry a target portion of a person's skin. In some examples the method may use an apparatus having a belt portion and a body portion. The method may include placing the belt portion against an abdominal area of a person so that the body portion covers the target portion of the person's skin, securing the belt portion against the abdominal area of the person to form a watertight seal between the apparatus and skin of the abdominal area, wherein water is prevented from passing through the watertight seal; and retaining, based at least in part on the securing, the body portion in a position covering the target portion of the person's skin.

In some examples of the method, the body portion may include a water-impenetrable exterior membrane. In some examples the method may further include shielding the target portion of the person's skin from water based at least in part on the water-impenetrable exterior membrane.

In some examples of the method, the apparatus may further include a body top end forming a first opening sized to receive a portion of the abdominal area therethrough, and a body bottom end forming at least one second opening sized to receive a portion of the person's body therethough. In some examples of the method, the placing may further include receiving the portion of the abdominal area through the first opening, and receiving the portion of the person's body through the at least one second opening. In some examples, the method may further include fastening, prior to the retaining, the body top end to the belt portion to form a liquid impermeable connection.

The foregoing has outlined some features of examples according to the disclosure in order that the detailed description that follows may be better understood. Additional features and steps will be described hereinafter. The conception and specific examples disclosed may be readily utilized as a basis for modifying or designing other structures and methods for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

FIG. 1 is a front view of one embodiment of an apparatus for keeping dry a portion of the abdominal area, in accordance with one or more aspects of the disclosure.

FIG. 2 is a back view of the embodiment of FIG. 1, in accordance with one or more aspects of the present disclosure.

FIG. 3 is a front perspective view of the embodiment of FIG. 1 showing an unfastened belt portion, in accordance with one or more aspects of the present disclosure.

FIG. 4 is a front perspective view of the embodiment of FIG. 1 showing the belt portion fastened, in accordance with one or more aspects of the present disclosure.

FIG. 5 is a perspective view of the embodiment of FIG. 1, shown in an environment of use, in accordance with one or more aspects of the present disclosure.

FIG. 6 is a back perspective view of the embodiment of FIG. 1, in accordance with one or more aspects of the present disclosure.

FIG. 7 is a back perspective view of another embodiment of an apparatus for keeping dry a portion of the abdominal area, in accordance with one or more aspects of the present disclosure.

FIG. 8 is a perspective view of another embodiment of an apparatus for keeping dry a portion of the abdominal area, in accordance with one or more aspects of the present disclosure.

FIG. 9 is a perspective view of the embodiment of FIG. 7, in accordance with one or more aspects of the present disclosure.

FIG. 10A is a front view of another embodiment of an apparatus for keeping dry a portion of the abdominal area, in accordance with one or more aspects of the disclosure.

FIG. 10B is a front view of an embodiment of belt for use with the apparatus of FIG. 10A, in accordance with one or more aspects of the disclosure.

FIG. 11A is a front perspective view of the embodiment of FIG. 10A in an environment of use worn by a person, in accordance with one or more aspects of the present disclosure.

FIG. 11B is a back perspective view of the embodiment of FIG. 10A in an environment of use worn by a person, in accordance with one or more aspects of the present disclosure.

FIG. 11C is a front perspective view of the embodiment in an environment of use worn by a person, in accordance with one or more aspects of the present disclosure.

FIG. 11D is a front perspective view of the embodiment in an environment of use worn by a person, in accordance with one or more aspects of the present disclosure.

### DETAILED DESCRIPTION

A few embodiments of the disclosure and improvements have been described. It should be understood that this description has been made by way of example, and that the invention is defined by the scope of the claims. Moreover, the drawings are provided in a utility application to support the claims and are not expected to give either photographic representation, or exact visual depictions.

Abdominal area may be defined herein as including, for example: at least a portion of the torso such as the chest and abdomen (including the stomach area) and/or the midsection and hip area.

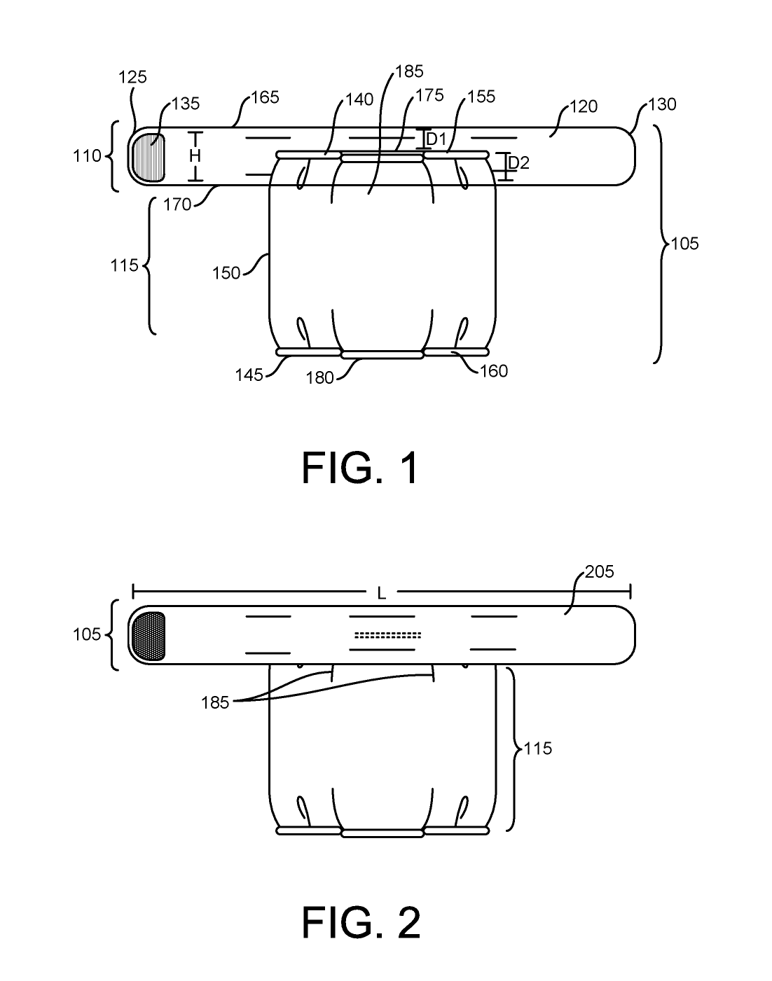

Turning to the figures, FIG. 1 is a front view of one embodiment 105 of an apparatus for keeping dry a portion of the abdominal area, in accordance with one or more aspects of the disclosure. For example, the apparatus embodiment 105 may help keep a portion of a person's skin dry, such as skin of the abdominal area. Such areas may, for example, have incisions, drains, tubes, etc., or other features that should stay dry to avoid infection and/or other negative consequences.

The apparatus 105 may include a belt portion 110 having an outer side 205 (as shown in FIG. 2) and an inner side 120 (as shown in FIG. 1). All or part of the belt portion 110 may be made of waterproof material. In some examples, the waterproof material may include synthetic rubber. In some examples, the synthetic rubber may be produced using processes involving the polymerization of chloroprene. In some examples, the polychloroprene may be waterproof neoprene, and for instance neoprene bonded sponge waterproof wetsuit fabric.

In some examples, the belt portion 110 may have an exterior that includes the waterproof neoprene and an interior that includes rubber. In some examples, the waterproof neoprene may be bonded with other materials—such as in some instances a polyester component, and in some instances also a spandex component. In some instances, the polyester component may be larger than the spandex component (for example, 92% Polyester/8% Spandex).

In some examples, the belt portion 110 may have a distal end 125 and a proximal end 130 opposite from the distal end 125. The belt portion 110 may be capable of two-way stretch—e.g., be configured to expand and contract in the both the direction of the distal end 125 and in the direction of the proximal end 130. In some examples, the belt portion 110 may also include a fastener 135 to fasten the distal end 125 to the proximate end 130. In some examples, the fastener 135 may incorporate (but is not limited to) hook and loop fasteners, such as for example, those associated with Velcro.

In some examples, the belt portion 110 may include a belt top end 165 and a belt bottom end 170 and a height H separating the belt top end 165 from the belt bottom end 170.

The apparatus embodiment 105 may also include a body portion 115. The body portion 115 may in some examples be tubular shaped at least on one end, and have a body top end 140 having a first opening 175, a body bottom end 145 having at least a second opening 180, and a body middle 150 connecting the body top end 140 and the body bottom end 145. The body portion 115 may be made of a liquid-impermeable material. For instance, in some examples the body portion 115 may be made of ripstop nylon fabric. In some examples, the body portion 115 may be made of different materials. In some examples, the body portion 115 may be made of any variety of waterproof fabric, including for example, disposal or reusable waterproof fabric. In some examples, 1 millimeter disposable Polyethylene fabric may be used as disposable waterproof fabric.

The body top end 140 of the body portion 115 may also be fastenable to the belt portion 110 at the adjacent first opening to form a liquid impermeable connection. In some examples, the body top end 140 may be fastenable to the belt bottom end 170 (or alternatively or in addition, to the belt top end 165, or at some point along the height H of the belt portion 110) to form the liquid impermeable connection therebetween.

In some instances, the body portion 115, and for example the body top end 140 thereof, may be stitched to the belt portion 110. In some examples, the body top end 140 and/or surrounding portions may be configured to fasten to the inner side 120 of the belt portion 110 so that the belt top end 165 lies a first distance D1 above the body top end 140, and the belt bottom end 170 lies a second distance D2 below the body top end 140. In some examples, the first distance D1 may be in the range of about 0.5 inches to about 3 inches and the second distance D2 may be in the range of about 0.5 inches to about 3 inches. The values of the first and second distances D1, D2 may depend at least in part of the height H and/or the size of the body top end 140, but in one example may each be about one inch.

In some examples, the belt portion 110 may be permanently fastened to the body portion 115 and in other examples the belt portion 110 may be temporarily and/or removably fastened to the body portion 115 or vice versa (e.g., the body portion 115 may be fastened to the belt portion 110). In some examples, the belt portion 110 may be permanently fastened to the body portion 115 using stitching.

In some examples, the body top end 140 may include elastic material 155. In some examples, the body bottom end 145 may include elastic material 160. In some examples, elastic material (e.g., 155 and 166) may be sewn into the body top end 140 and body bottom end 145. In some examples, at or near the body top end 140 of the body portion 115, gathers 185 may form, for example, due at least in part or primarily or entirely to the elastic material 155. Gathers 185 may increase the risk of water penetration. Alternatively, the elastic material 160 may be configured to prevent gathers, by for example providing a more uniform body top end 140 to which the belt portion 110 may fasten. In some examples the elastic material 155, 160 may be sewn into the top and bottom ends.

FIG. 2 is a back view of the embodiment 105 of FIG. 1, in accordance with one or more aspects of the present disclosure. As described above, the belt portion 110 may overlap the body top end 140, and in some examples overlap in such a manner as to cover the gathers 185 with the outer side 205 of the belt portion 110, so as to prevent and/or decrease the probability of water penetration due at least in part to the gathers 185.

In some examples the belt portion 110 may be made of different materials that may also overlap the body top end 140 and for example cover the gathers 185. For example, in some variations a wide, thin “rubber band” sheet may be used, and/or waterproof material with flexible and stretchy features. In some aspects, however, the belt portion 110 may be able to stretch sufficiently tight to create a seal with the skin of a person (as shown in greater detail in FIG. 4—for example, around the top portion of the abdomen).

In some examples, the length L of the belt portion 110 may be in the range of about 40-75 inches, and 54-60 inches in some examples. The belt portion 110 may have a thickness in the range of about 0.1 millimeters to about 7 millimeters. The body portion 115 may also have a variety of dimensions and sizes (for example according to a person's body weight, size, and shape, etc.).

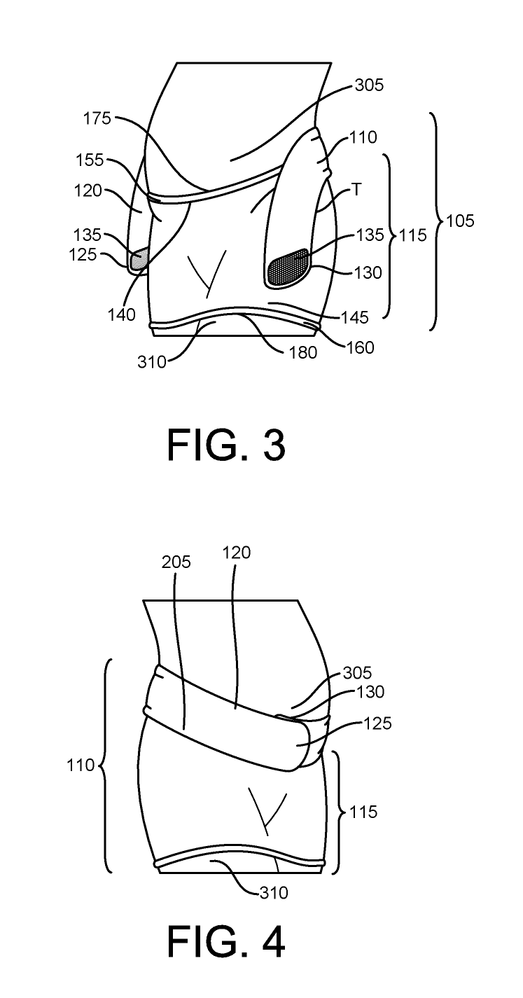

FIG. 3 is a front perspective view of the embodiment of FIG. 1 showing an unfastened belt portion 110, in accordance with one or more aspects of the present disclosure. In some examples, the belt portion 110 may have the thickness T in the range of about 0.25 millimeters to about 3 millimeters, and in some examples a sponge thickness of 2 mm or 1 mm.

The body top end 140 of the belt portion 110 may form a first opening 175 that may be sized to receive a portion of the abdominal area 305. The body portion 115 may also be configured to cover at least a portion of the abdominal area 305. The at least a portion of the abdominal area 305 covered by the body portion 115 may include an incision.

As mentioned with regard to FIG. 1, in some examples the apparatus 105 may also include a fastener 135 to fasten the distal end 125 to the proximate end 130 while the belt portion 110 is wrapped around the abdominal area 305 (as shown in FIG. 4). The fastener 135 may be located at or near the distal end 125 and/or at or near the proximate end 130. In some examples, the belt portion 110 may have a length L between the distal end 125 and the proximate end 130 fastened to the distal end 125, and in some examples the length L may be adjustable for wrapping the belt portion 110 around different sized abdominal areas. As mentioned above, in some examples, the length L may be in the range of about 40-75 inches, and in other examples the length L may be outside that range. In some examples, the fastener(s) 135 may include hook and loop fasteners.

The second opening 180 of the body bottom end 145 may receive at least a portion of a person's body 310 therethrough. The portion of the person's body 310 received through the second opening 180 may vary, for example, according to placement, size, and dimensions of the apparatus 105.

The elastic material 155 and 160 may press against a portion of the abdomen 305 and another portion of a body 310 so as to assist in substantially retaining a position of the body portion 115 against the person (i.e., keeping the apparatus 105 “in place”).

FIG. 4 is a front perspective view of the embodiment of FIG. 1 showing the belt portion 110 fastened, in accordance with one or more aspects of the present disclosure. The belt portion 110 may fasten, for example, by the distal end 125 fastening to the proximal end 130 using a fastener 135 and/or fasteners 135 (incorporating, e.g., hook and loop fasteners) on one or both of the distal end 125 and proximal end 130.

As shown in FIG. 4, the apparatus 105 may be configured for placement so that the inner side 120 of the belt portion 110 may face against an abdominal area 305 of a person's body, so that the body portion 115 may cover a portion of the abdominal area 305 having for example an incision. The belt portion 110 may be placed against a variety of spots, points, heights, etc. of an abdominal area 305 of a person's body, so for example that the body portion 115 (and/or at least a portion of the belt portion 110) may cover a different at least a portion of the abdominal 305 and/or other portions of the person's body 310 (depending for example on where an incision is located or otherwise has need for waterproof protection). For example, the belt portion 110 may be placed differently (e.g., higher) in the abdominal area 305 than shown in FIG. 4.

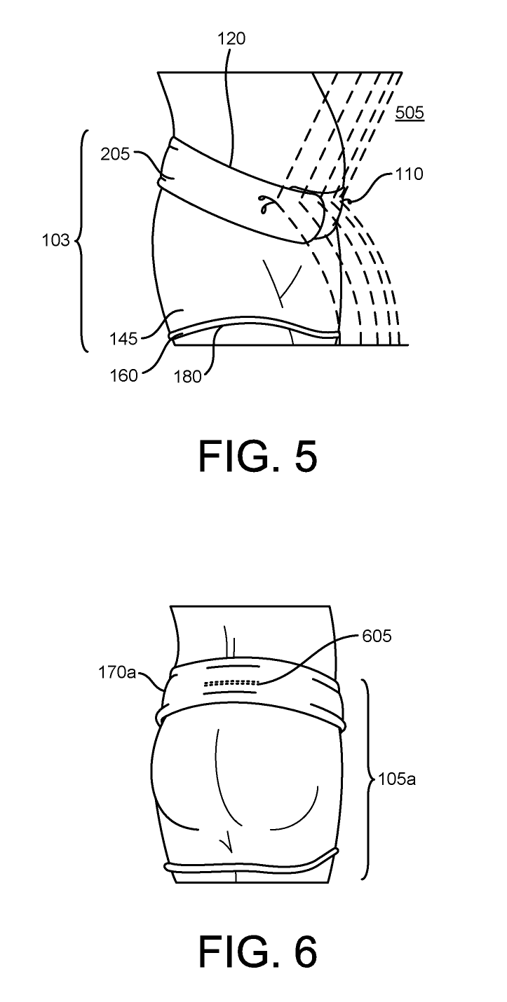

FIG. 5 is a perspective view of the embodiment of FIG. 1, shown in an environment of use, in accordance with one or more aspects of the present disclosure. FIG. 5 is a perspective view of the apparatus 105, and may help illustrate for example the waterproof properties of the apparatus 105.

As shown in FIG. 5, the apparatus 105 may be configured for placement of the inner side 120 against an abdominal area 305 of a person's body to prevent water 505 from passing between the inner side 120 and the skin of the abdominal area 305. In some examples, the belt portion 110 may create a seal around the a(n e.g., top) portion of the abdominal area 305. In some examples, the belt portion 110 may apply a constricting force against the abdominal area 305 sufficient to prevent water from passing under the belt portion 110, yet not so great a force as to generate discomfort and/or restrict blood circulation. The material of the apparatus's 105 belt portion 110 may assist in achieving certain benefits described herein, e.g., forming a seal, preventing the entering of water 505, not abrading skin or restricting blood circulation or otherwise creating discomfort.

Thus, in some examples, when showering the water 505 may not pass between the inner side 120 of the belt portion 110 and the skin of the abdominal area 305. But rather, for example, some of the water 505 may run off or be repelled by the outer side 205 of the belt portion 110 and fall down. Moreover, for example, gravity (and/or the elastic material 160) may prevent the water 505 from entering the at least one second opening 180 of the bottom end 145.

FIG. 6 is a back perspective view of the embodiment of FIG. 1, in accordance with one or more aspects of the present disclosure. For example, FIG. 6 may show the stitching 605 for an embodiment of the apparatus 105a having a belt portion 110a that is permanently attached by being sewn on.

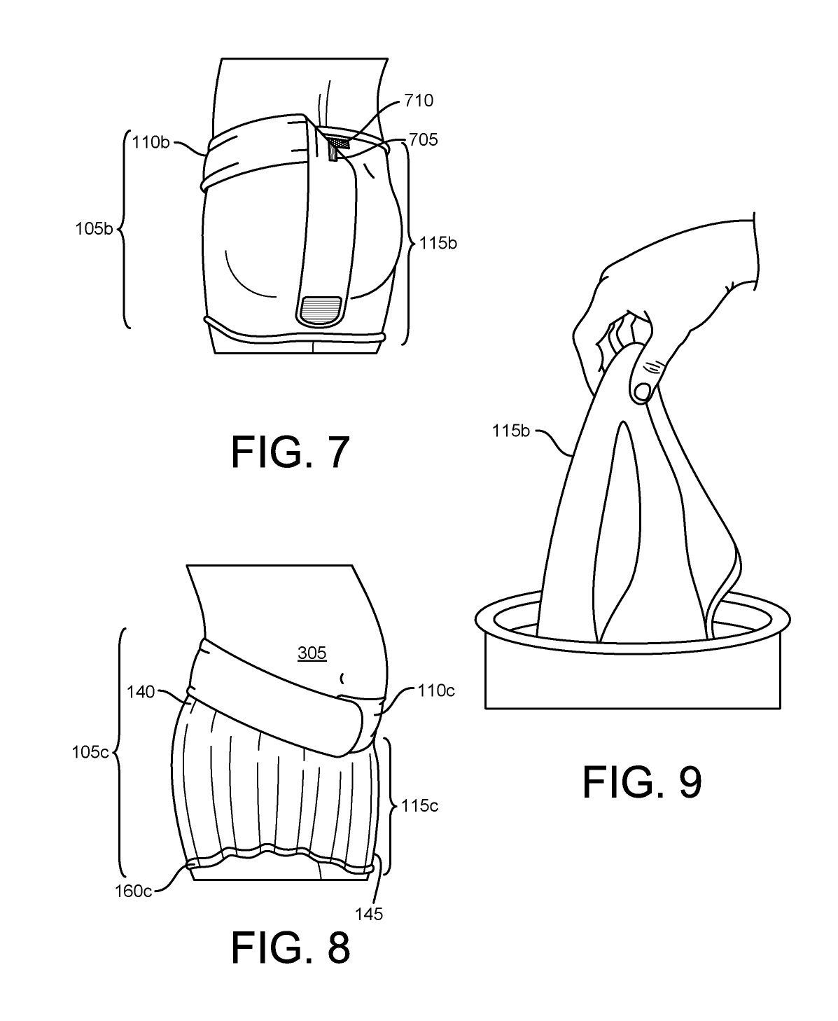

FIG. 7 is a back perspective view of another embodiment of an apparatus 105b for keeping dry a portion of the abdominal area, in accordance with one or more aspects of the present disclosure. FIG. 7 may show a detachable belt portion 110b partially detached from the tube portion 115b. The belt portion 110b may attach and detach to the body portion 115b using for example belt portion hook and loop fasteners 705 and/or body portion hook and loop fasteners 710. Some examples of a detachable belt portion 110b may make it easier to remove and dispose of a body portion 115b that is replaceable.

FIG. 8 is a perspective view of another embodiment of an apparatus 105c for keeping dry a portion of the abdominal area 305, in accordance with one or more aspects of the present disclosure. The belt portion 110c may be placed and/or configured for placement around different portions of the body, depending for example on the location of an incision. In addition, the shape of the body portion 115c may be placed and/or configured for placement to cover different areas of a person's body (including different sizes of areas). For example, the shape of different variations of the body portion 115c may be may configured to cover the “mid-section” and higher than that shown in FIG. 8.

Moreover, the shape of the body portion 115c may not necessarily “cling” or be “form-fitted” to part of a person's body. For example, in some aspects the body portion 115c may be more loose fitting (for example, in some variations, like a hollow tube). In addition, in some examples, the loose fitting body portion 115c may be cinched at the top and bottom, for example with elastic material (155 not shown) and 160c. Such cinching may assist in preventing the apparatus 105c and/or the body portion 115c from falling off. For example, when the belt portion 110c is unfastened and/or tightened, the cinching may allow the apparatus 105c to remain in a relatively static position while the belt portion 115c may be tightened. Thus, the top portion 140 and the bottom portion 145 may be cinched, and the belt portion 110c may be at least partially positioned above the top of the body portion 115c and affixed to the body portion 115c either permanently or removably.

FIG. 9 is a perspective view of the embodiment of FIG. 7, in accordance with one or more aspects of the present disclosure. FIG. 9 may illustrate, for example, the disposability of the body portion 115b of the apparatus 105b. For example, after one or several uses, the body portion 115b may begin to exhibit wear and tear and the waterproof membrane thereof may risk becoming compromised. In addition, and also by way of example, in certain settings (such as hospitals), the belt portion 110b, which may in some examples be made of a more complex and potentially expensive material (e.g., as may be the case for a stretch neoprene belt) may be preserved and/or washed or sanitized for reuse, while the body portion 115b may be intended for a one time (or several-times) reuse for just a single particular patient. Thus the body portion 115b may be properly discarded and replaced with a new body portion 115b for use with the belt portion 110b. In addition, different users of different sizes and dimensions may share use of the same belt portion 110b, but use different body portions 115b customized or better suited to their particular body shape and or intended use (e.g., a body potions 115b configured to protect certain and potentially different areas where an incision and/or infected area may be located). The material used for a disposable body portion 115b may be selected-suited for such disability, as opposed to material for a body portion intended by longer use.

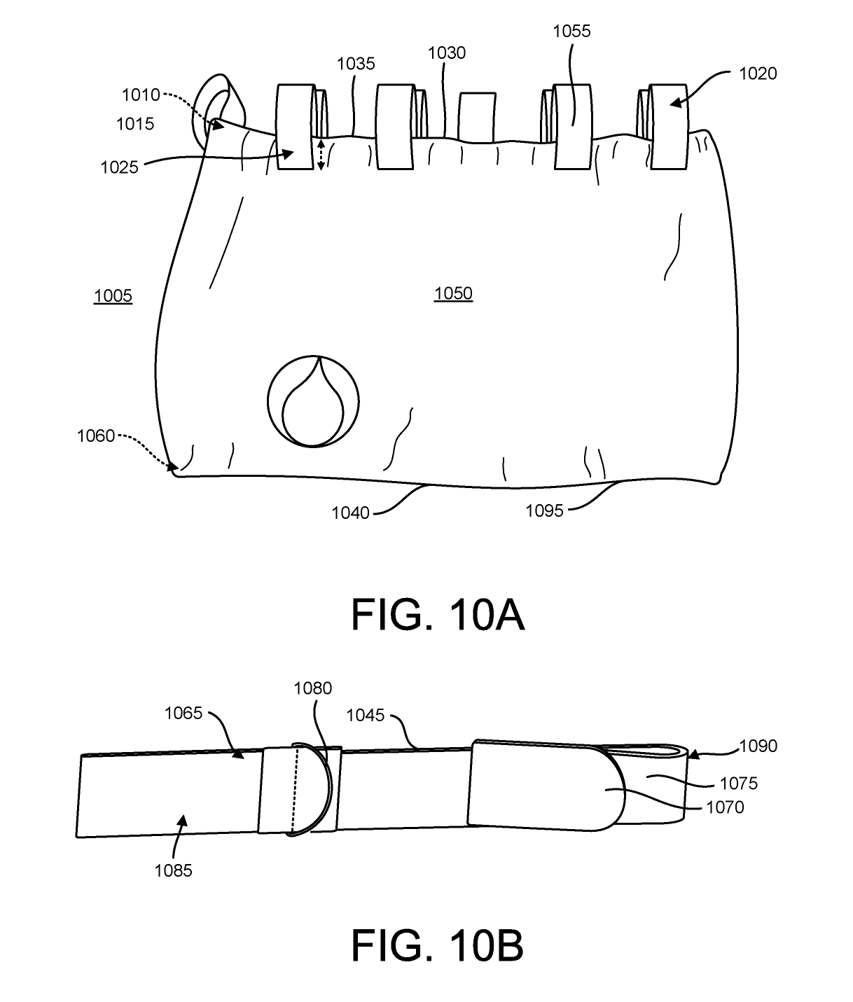

FIG. 10A is a front view of another embodiment of an apparatus for keeping dry a portion of the abdominal area, in accordance with one or more aspects of the disclosure. For example, the apparatus embodiment 1005 may help keep a portion of a person's skin dry, such as skin of the abdominal area. Such areas may, for example, have incisions, drains, tubes, etc., or other features that should stay dry to avoid infection and/or other negative consequences.

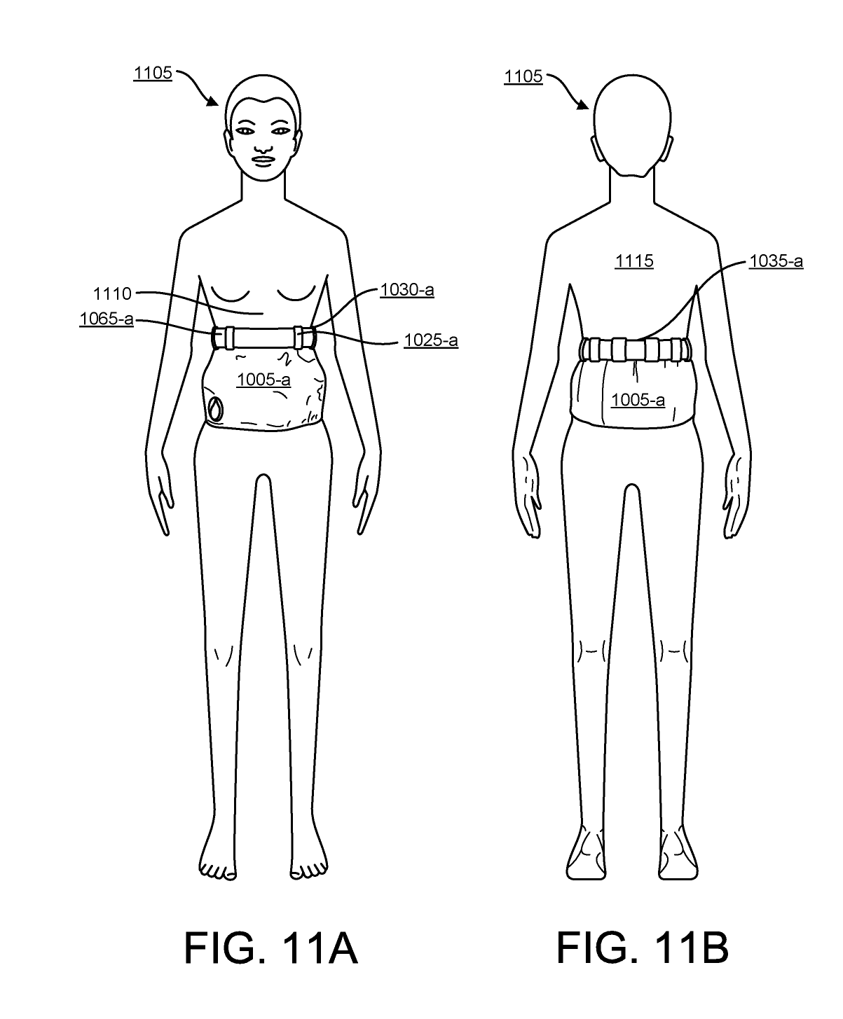

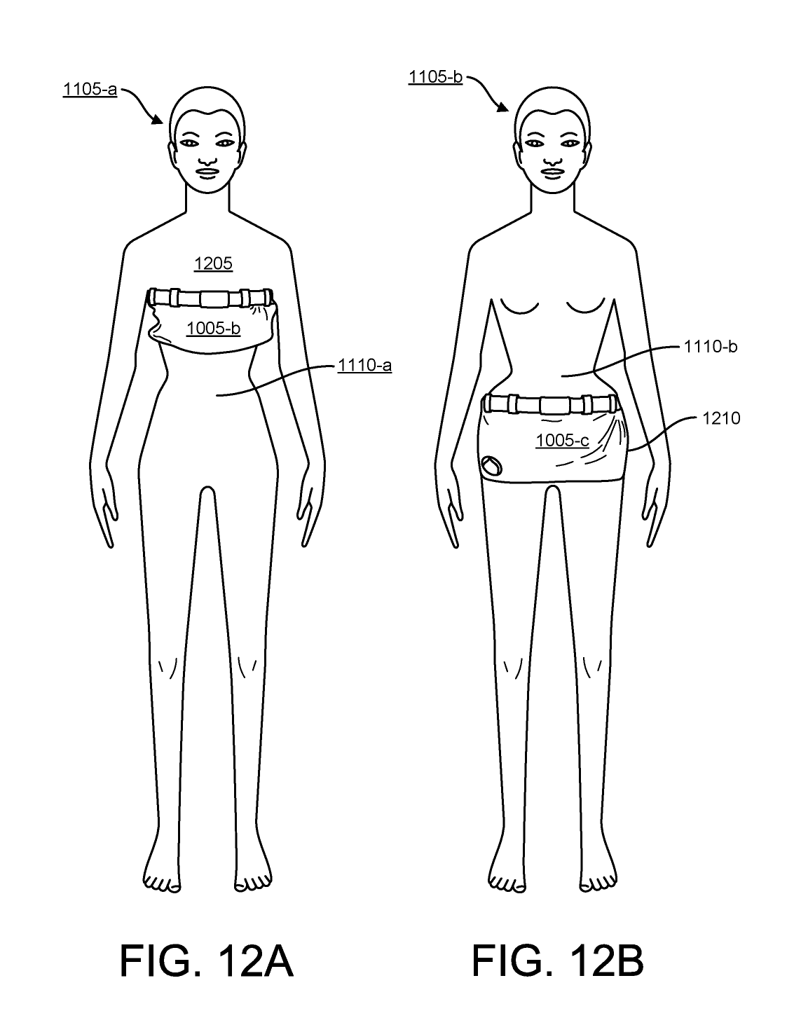

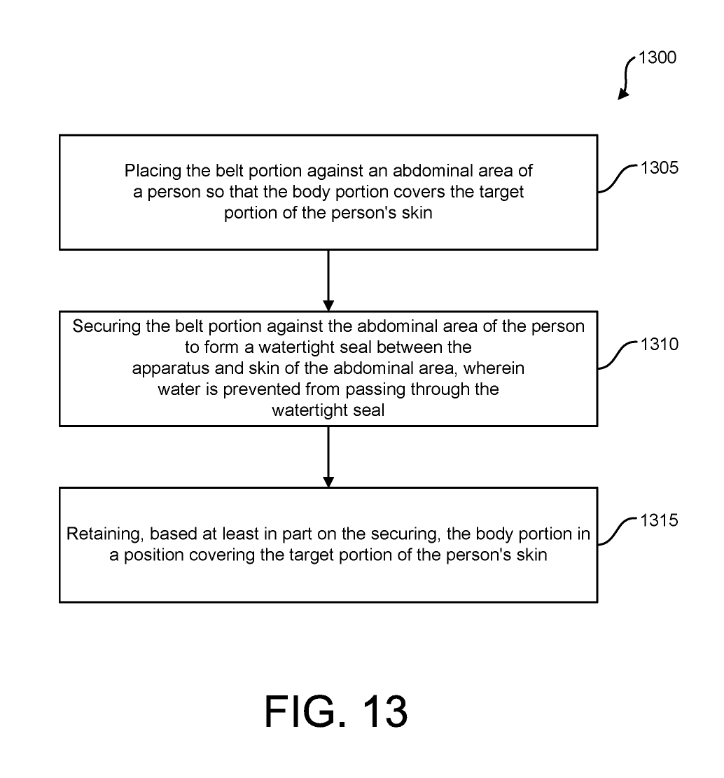

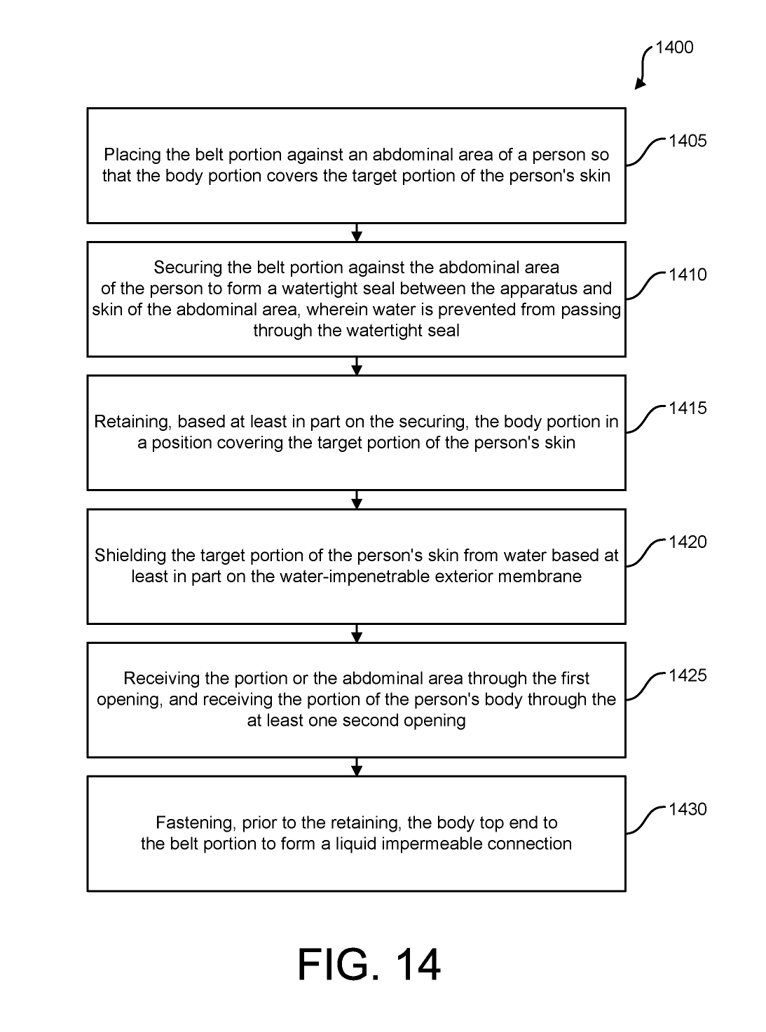

The apparatus 1005 may include an upper retaining portion 1010, to assist in retaining the apparatus 1005 against a body (e.g., the abdominal area of a body—as shown in FIGS. 11A-11D). In some embodiments, the upper retaining portion 1010 may include a top elastic member 1015. In some embodiments, the upper retaining portion 1010 may include a belt attacher 1020. In some examples, the belt attacher 1020 may include a plurality of belt loops 1025.

The apparatus embodiment 1005 may also include a body portion 1025. The body portion 1025 may in some examples be shaped to cover a part of the body, and may have a body top end 1030 having a first opening 1035, a body bottom end 1040 having at least a second opening 1045, and a body middle 1050 connecting the body top end 1030 and the body bottom end 1040. The embodiment 1005 may be tubular shaped at one or more of the body top end 1030 and the body bottom end 1040.

In some examples, the body top end 1030 may include top elastic member 1015. In some examples, a portion 1055 of each of the plurality of belt loops 1025 may be fastened to the body top end 1030. In one embodiment the portion 1055 of each of the plurality of belt loops 1025 may extend about 0.5 inches (e.g., in the range of 0.3-0.7 inches) below the top elastic member 1015 of the body top end 1030. In some examples, the body bottom end 1040 may include a bottom elastic member 1060. In some examples the top elastic member 1015 and/or the bottom elastic member 1060 may be sewn into or otherwise secured to the body top end 1030 and body bottom end 1040.

The body portion 1025 may be made of a liquid-impermeable material. For instance, in some examples the body portion 115 may be made of waterproof material that may nevertheless allow heat to escape (e.g., is “breathable”), and have a degree of elasticity (e.g., “stretch” such as a cross-grain stretch) fabric, such as e.g., polyurethane laminate (“PUL”) material. In some examples the PUL material may be made of a durable material, such as for example that provided by some combinations of polyester and polyurethane. In some examples, the combination may be in a range of 35% to 70% polyester and 30% to 65% polyurethane (e.g., 55% polyester and 45% polyurethane). In some examples, the material may be made of other substances in addition to or alternative to polyester and polyurethane. In some examples the material may be laminated knit fabric. In some examples the thickness of the material may be in a range of 0.3 millimeters to 3 millimeters (e.g., 1 millimeter). In some examples, the material may be compliant with the Consumer Product Safety Improvement Act (CPSIA).

FIG. 10B is a front view of an embodiment of a belt 1065 for use with the apparatus of FIG. 10A, in accordance with one or more aspects of the disclosure. The belt 1065 may fasten to or attach to or otherwise be used with the apparatus embodiment 1005, and in particular to/with the upper retaining portion 1010 thereof, (and e.g., the belt attacher 1020 thereof) to assist in retaining the apparatus 1005 against a body. For example, in some embodiments the belt 1065 may slide through the plurality of belt loops 1025. Then, in some examples, the belt 1065 may be tightened by ways known by the person having ordinary skill in the art. In some examples, a first end 1070 of the belt 1065 may fasten to a second opposite end 1075 of the belt 1065. For example, in one embodiment the first end 1070 end (e.g., several inches thereof—for example, four inches) may include un-broken loop (“UBL”) fabric that may include “hook” Velcro features 1095, while the second end 1075 may include UBL fabric that may include “loop” Velcro features, which may allow the first end 1070 and the second end 1075 to attach to each other. In some embodiments the belt 1065 may include a “D ring” 1080 to facilitate tightening (and securing in place) the belt 1065. Other embodiments may include, for example, a ladder lock slider plastic buckle fastener to also facilitate tightening and securing.

The belt 1065 may have a front side 1085 and a back side 1090. All or part of the belt portion 1065 may be made of waterproof material. In some embodiments, the waterproof material may include waterproof neoprene—e.g., on the front side 1085 of the belt 1065. In some examples, the front side 1085 of the belt 1065 may include UBL Velcro 1090 for attaching with the first end 1070 and/or the second end 1075 (e.g., the front side 1085 may include UBL loop features to attach with the first end 1070), to provide an adjustable secure fit. In some examples, the front side 1085 may also be referred to as an outer side. In some examples a back side (not shown in detail, and which may also be referred to as an inner side) may include material for forming a waterproof seal with skin of a body.

In some examples, the material of the belt 1065 may be mostly neoprene, and in some examples 100% neoprene. In some examples the material of the belt 1065 may include a super stretch neoprene sheet. In some examples, a first material such as the neoprene may be laminated with a second material (having, for example, elastic features), such as nylon and/or UBL material. In one example, such second material of the belt 1065 may include 100% four-way super-stretch nylon and U.B.L., which may provide elasticity and durability. In some examples, the belt 1065 interior (not shown) may include or be made of soft nylon, which may provide for a soft comfortable fit.

FIG. 11A is a front perspective view of the embodiment 1005-a of FIG. 10A in an environment of use worn by a person 1105, in accordance with one or more aspects of the present disclosure. In some examples, as previously mentioned, at least portions of the plurality of belt loops 1025-a (in pertinent embodiments having such belt loops) may extend below the body top end 1030-a (and the top elastic member 1015 thereof). With the belt 1065-a placed through the plurality of belt loops, the belt 1065-a may assist in creating a waterproof seal against the skin of the body 1110 and thus may seal over the body top end 1030-a (and e.g., the top elastic member 1015 thereof).

FIG. 11B is a back perspective view of the embodiment 1005-a of FIG. 10A in an environment of use worn by the person 1105, in accordance with one or more aspects of the present disclosure. In some examples, the belt 1065-a may also assist in creating the waterproof seal against the skin of the back of the person's body 1115 and all the way around the body 1110 of the person where the belt 1065-a is positioned, and over the body top end 1030-a (and e.g., the top elastic member 1015 thereof), thereby preventing water from entering the first opening 1035-a of the apparatus embodiment 1005-a.

FIG. 12A is a front perspective view of an embodiment 1005-b in an environment of use worn by a person 1105-a, in accordance with one or more aspects of the present disclosure. The apparatus embodiment 1005-b may be placed at various different parts of the body 1110-a of the person 1105-a. In some examples, different apparatus embodiments may be configured for placement at different body parts—e.g., body parts relating to the abdominal area. For example, one apparatus embodiment 1005-b may be placed against the torso 1205 to, for example, prevent water from irritating or contaminating an incision located at or near the area of the torso 1205.

FIG. 12B is a front perspective view of the embodiment 1005-c in an environment of use worn by a person 1105-b, in accordance with one or more aspects of the present disclosure. As mentioned above, in some examples, different apparatus embodiments may be configured for placement at different body parts of the body 1110-b of the person 1105-b—e.g., body parts relating to the abdominal area. Alternatively or in addition, a single apparatus embodiment may be configured for placement at different body parts relating to the abdominal area. For example, another apparatus embodiment 1105-b may be placed against or over the waist or hip area 1210 to, for example, prevent water from irritating or contaminating an incision located at or near the area of the waist or hip area 1205.

FIG. 13 is a flow diagram illustrating one embodiment of a method 1300 for keeping dry a target portion of a person's skin. In some examples the method may use an apparatus having a belt portion and a body portion. At block 1305, method 1300 may include placing the belt portion against an abdominal area of a person so that the body portion covers the target portion of the person's skin. At block 1310, method 1300 may include securing the belt portion against the abdominal area of the person to form a watertight seal between the apparatus and skin of the abdominal area, wherein water is prevented from passing through the watertight seal. At block 1315, method 1300 may include retaining, based at least in part on the securing, the body portion in a position covering the target portion of the person's skin.

FIG. 14 is a flow diagram illustrating another embodiment of a method 1400 for keeping dry a target portion of a person's skin. At block 1405, method 1400 may include placing the belt portion against an abdominal area of a person so that the body portion covers the target portion of the person's skin. At block 1410, method 1400 may include securing the belt portion against the abdominal area of the person to form a watertight seal between the apparatus and the skin of the abdominal area, wherein water is prevented from passing through the watertight seal. At block 1415, method 1400 may include retaining, based at least in part on the securing, the body portion in a position covering the target portion of the person's skin.

In some examples of the method, the body portion may include a water-impenetrable exterior membrane. At block 1420, method 1400 may include shielding the target portion of the person's skin from water based at least in part on the water-impenetrable exterior membrane. In some examples, the apparatus may further include a body top end forming a first opening sized to receive a portion of the abdominal area therethrough, and a body bottom end forming at least one second opening sized to receive a portion of the person's body therethough. At block 1425 of the method 1400, the placing may further include receiving the portion of the abdominal area through the first opening, and receiving the portion of the person's body through the at least one second opening. At block 1425, the method 1400 may further include fastening, prior to the retaining, the body top end to the belt portion to form a liquid impermeable connection.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

In addition, the above-described apparatuses and methods are considered that of some embodiments only. It is therefore understood that the embodiments shown in the figures below and described above are merely for illustrative purposes and not intended to limit scope. Alterations and modifications, therefore, and such further applications as would occur to those skilled in the relevant art(s), are also contemplated. For example, in some embodiments the belt portion may be the body portion, and the body portion may be placed on the abdominal area by wrapping around and then fastening.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

I claim:

1. An apparatus for keeping dry a portion of a person's skin, comprising:
a belt portion having an outer side and an inner side, the belt portion being configured for placement of the inner side against an abdominal area of a person's body to prevent water from passing between the inner side and the abdominal area, the belt portion comprising waterproof material; and
a body portion configured to cover at least a portion of the abdominal area, comprising:
a body top end forming a first opening sized to receive a portion of the abdominal area, the body top end being fastenable to the belt portion adjacent to the first opening to form a liquid impermeable connection;
a body bottom end having at least one second opening sized to receive a portion of the person's body;
a body middle connecting the body top end and the body bottom end, and comprising liquid-impermeable material.

2. The apparatus of claim 1, wherein the body portion is tubular shaped and the portion of the person's body the second opening is sized to receive is another portion of the abdominal area.

3. The apparatus of claim 2, wherein the belt portion and the body portion are further configured for placement against the abdominal area at at least one of: a torso, a midriff, a waist, and an abdomen of the person's body.

4. The apparatus of claim 3, wherein the belt portion comprises a distal end, a proximal end opposite from the distal end, and a fastener for fastening the distal end to the proximate end.

5. The apparatus of claim 4, wherein the belt portion is elastically deformable along an axis defined by a length of the belt between the distal end and the proximal end.

6. The apparatus of claim 1, wherein the body top end is fastenable to the belt portion along the body top end opening to form a liquid impermeable connection therebetween.

7. The apparatus of claim 6, wherein the belt portion is fastenable to the belt portion so that a top end of the belt portion lies a first distance above the body top end and a bottom end of the belt portion lies a second distance below the body top end.

8. The apparatus of claim 1, wherein the belt portion is removably fastenable to the body portion.

9. The apparatus of claim 1, wherein the waterproof material of the belt portion comprises synthetic rubber.

10. The apparatus of claim 9, wherein the synthetic rubber comprises polychloroprene.

11. The apparatus of claim 10, wherein the polychloroprene comprises 100% neoprene.

12. The apparatus of claim 1, wherein the belt portion comprises a first material and a second material, the second material being distinct from the first material.

13. The apparatus of claim 12, wherein the first material is associated with the outer side of the belt portion, and the second material is associated with the inner side of the belt portion.

14. The apparatus of claim 12, wherein the belt portion comprises an outer surface and an interior, and wherein the first material is associated with the outer surface of the belt portion, and the second material is associated with the interior of the belt portion.

15. The apparatus of claim 1, wherein the body bottom end comprises elastic material, or the body top end comprises elastic material, or both.

16. The apparatus of claim 1, wherein the body portion comprises polyurethane laminate.

17. A method for keeping dry a target portion of a person's skin using an apparatus having a belt portion and a body portion, comprising:
placing the belt portion against an abdominal area of a person so that the body portion covers the target portion of the person's skin;
securing the belt portion against the abdominal area of the person to form a watertight seal between the apparatus and skin of the abdominal area, wherein water is prevented from passing through the watertight seal; and
retaining, based at least in part on the securing, the body portion in a position covering the target portion of the person's skin.

18. The method of claim 17, wherein the body portion comprises a water-impenetrable exterior membrane, and further comprising:
shielding the target portion of the person's skin from water based at least in part on the water-impenetrable exterior membrane.

19. The method of claim 17, wherein the body portion comprises a body top end forming a first opening sized to receive a portion of the abdominal area therethrough, and a body bottom end forming at least one second opening sized to receive a portion of the person's body therethough, and wherein the placing further comprises:
receiving the portion of the abdominal area through the first opening; and
receiving the portion of the person's body through the at least one second opening.

20. The method of claim 19, further comprising:
fastening, prior to the retaining, the body top end to the belt portion to form a liquid impermeable connection.
